(19) United States
(12) Patent Application Publication
(10) Pub. No.: US 2011/0217364 A1
(43) Pub. Date: Sep. 8, 2011

(54) CONJUGATED SURAMIN AMINO COMPOUNDS FOR MEDICAL CONDITIONS

(76) Inventor: Sammy O. Opiyo, Nairobi (KE)

(21) Appl. No.: 13/057,643

(22) PCT Filed: Jul. 10, 2009
(86) PCT No.: PCT/KE09/00019

§ 371 Date: Feb. 4, 2011

(30) Foreign Application Priority Data
Aug. 4, 2008 (KE) KE/2008/000782

(51) Int. Cl. A61K 31/185; A61K 9/127; A61K 31/198; A61K 31/195; A61P 35/00; A61P 37/00; A61P 31/18; A61P 3/00; A61P 33/02

(52) U.S. Cl. 424/450; 514/577; 514/564; 514/565; 514/562

(57) ABSTRACT

A modification of pharmacokinetics of suramin by use of amino compounds in modification of lipophilicity and protein binding characteristics for tackling the physico-chemical properties of suramin that gives it poor distribution in the body; formulating the resulting product in a form that leads to targeting body fluids like the lymphatic system when administered in a specific way; and determining the route of administration that maximizes targeting with a view to reducing the dosage so as to limit toxicity, hence increase tolerance by patient.

### RELATED APPLICATIONS

The present application is a National Stage of International Application No. PCT/KE2009/000019, filed Jul. 10, 2009, which claims the benefit of Kenya Provisional Application Serial No. KE/P/08/00782, filed on Aug. 4, 2008, the contents of each being incorporated by reference herein in their entirety.

### TECHNICAL FIELD

The present invention relates generally to a modification of pharmacokinetics of suramin and particularly to the use of amino compounds in modification of lipophilicity and protein binding characteristics.

### BACKGROUND

Suramin was introduced in 1920 for treatment of trypanosomiasis. The original formulation which is still the standard used currently is as sodium salt that is prepared aseptically and dissolved in water just prior to use for injection.

Over the time suramin has been tried in various conditions including cancers, HIV/AIDS, and immunoregulatory disorders. However, in all those trials it has not progressed to clinical use level due to various challenges relating to its pharmacokinetics and toxicity.

### SUMMARY

The purpose of the present invention is therefore to create conjugates and derivatives of suramin with characteristics that when formulated and administered appropriately would reduce or eliminate these challenges.

The specific conjugates and salts are those of amino compounds. It has been demonstrated in various experiments and trials that suramin and its derivatives have a very high potential in the management of various conditions comprising viral infections, metabolic disorders, protozoal infections, and the like both in humans and animals.

It has again been demonstrated that suramin and derivatives are effective antivirals, anticancers, anti neoplastics, antiprotozoals, strong metabolic and immunomodulators etc., but they have limited use due to the difficulties already demonstrated.

In all these cases, the results of clinical trials have been disappointing as invitro results have not been translated into the desired clinical response in vivo, except in trypanosomiasis. All these have been compounded by high toxicity that limits long-term administration, and poor distribution that leads to limited availability for specific site activity. The latter leads to necessity to use high dosage that just increases the toxicity. As a result, a lot of work has been done to address these problems and have taken various dimensions.

It is in this regard that this particular invention intends to address the above shortfalls by: first, tackling the physico-chemical properties of suramin that gives it poor distribution in the body; second, formulating the resulting product in a form that leads to targeting body fluids like the lymphatic system when administered in a specific way; and third, determining the route of administration that maximizes targeting with a view to reducing the dosage so as to limit toxicity, hence increase tolerance by patient.

The overall goal is delivery of the product in amounts that is sufficient to give the desired effects at reduced or minimal side effects.

It is an object of the present invention, therefore, to create the conjugates and derivatives by use of amino compounds that have increased lipophilicity.

It is another object of the present invention to formulate the conjugates and derivatives.

It is yet another object of the present invention to administer the formulation appropriately to achieve a specific pharmacokinetics profile.

Further objects of the invention will become apparent to those skilled in the, art from examination of the following detailed description of the invention when taken in conjunction with the appended claims and figures.

### BRIEF DESCRIPTION AND DRAWINGS

FIG. 1 depicts the chemical structure of suramin;

FIG. 2 depicts the chemical structure of L-lysine; and

FIG. 3. depicts the chemical structure of L-arginine.

### DESCRIPTION

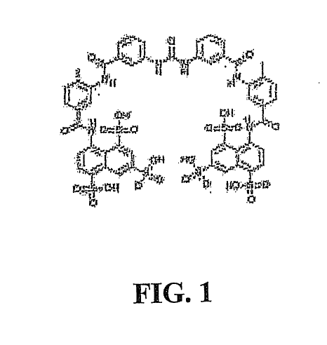

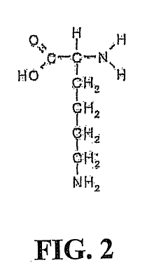

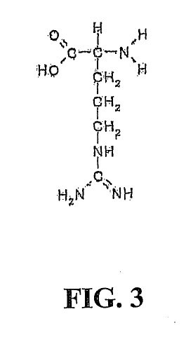

Depending on the amino compound in question, Suramin (depicted in FIG. 1), reacts spontaneously with the amino compound to form a different compound/conjugate with a different characteristic. Two of these amino compounds include L-lysine, depicted in FIG. 2, and L-arginine, depicted in FIG. 3. As an example, when Suramin was mixed in a vessel with 1-lysine HCl and warmed, chromatograms were obtained. Further characterization will be performed on IR to determine the nature of the compounds in this mixture.

Otherwise it is expected that the reaction will be:

Example: Suramin sodium+6[L-arginine hydrochloride]=Suramin L-arginate+6[Sodium Chloride].

Example: Suramin Hexasodium+6[L-Lysine Hydrochloride]→Suramin L-lysinate+6[Sodium Chloride].

This formation of a stable compound is supported by the fact that during HPLC analysis by ion pairing chromatography, a stable compound is formed that is lipophylic.

These hypotheses will be tried via IR spectroscopy.

Example: Extraction of Suramin from plasma samples using Tetrabutylammonium hydrogen sulfate [TBAHS] as the ion pairing reagent done by Kassack M. and Nickel P. [J Chromatogr B Biomed Appl. 1996 Nov. 15; 686[2]:274-84.

Example: Ion pairing HPLC method of suramin pharmacokinetics profile determination by J. M. Collins etal in 1986. Here Triethanolamine was used as the ion pair.

1-5. (canceled)

6. A method of managing medical conditions, comprising:
administering, to a human, an effective dose of conjugates and derivatives of suramin.

7. The method of claim 6, wherein administering the effective dose of conjugates and derivatives comprises delivery by one or more of sublingual, buccal, intraperitoneal, rectal, oral, and intrapulmonary modes.

8. The method of claim 6, further comprising formulating said conjugates and derivates for the human, wherein formulations of said conjugates and derivates comprise tablets, liposomal preparations, sterile injections, and suspensions.

9. A method of treatment, comprising:
administering, to a human, an effective dose of one or more of lysine, arginine, and other amino compounds to prevent an administration of an effective dose of suramin from resulting in serum albumin binding in the human.

10. The method of claim 9, wherein the administration of the effective dose of suramin includes derivatives and analogues of suramin.

11. The method of claim 9, wherein the other amino compounds provide an acidic moiety being carboxylic and sulphonic.

12. The method of claim 9, further comprising using the method of treatment for management of one or more medical conditions.

13. The method of claim 12, wherein the medical conditions comprise one or more of HIV/AIDS, cancer, trypanosomiasis, and metabolic disorders.

14. The method of claim 9, further comprising:
formulating a resulting product of the one or more of lysine, arginine, and other amino compounds into a form that leads to targeting body fluids including the lymphatic system of the human; and
determining a route of administration to maximize targeting with a view to reducing a dosage of the resulting product so as to limit toxicity, thereby increasing tolerance of the resulting product in the human.

15. A method of treatment, comprising
administering an effective dosage of a suramin product to a human; and
using one or more of lysine, arginine, and other amino compounds to protect the effective dosage of the suramin product from causing serum albumin binding in the human;
wherein the effective dosage of the suramin product is about 1.5 mg/kg body weight per day.

16. The method of claim 15, wherein the effective dosage of the suramin product comprises derivatives and analogues of suramin.

17. The method of claim 15, wherein administering the effective dosage of the suramin product is provided by subliginually or by any other acceptable route.
